(19) United States
(12) Patent Application Publication
(10) Pub. No.: US 2024/0299005 A1
(43) Pub. Date: Sep. 12, 2024

(54) CONTROL APPARATUS, CONTROL METHOD, AND CONTROL PROGRAM

(71) Applicant: FUJIFILM Corporation, Tokyo (JP)

(72) Inventors: Hisatsugu HORIUCHI, Kanagawa (JP); Lisako NOBUYAMA, Kanagawa (JP); Masataka SUGAHARA, Kanagawa (JP); Koji TANINAI, Kanagawa (JP)

(73) Assignee: FUJIFILM Corporation, Tokyo (JP)

(21) Appl. No.: 18/596,589

(22) Filed: Mar. 5, 2024

(30) Foreign Application Priority Data
Mar. 10, 2023 (JP) 2023-038161

(51) Int. Cl. A61B 8/00; A61B 6/46; A61B 6/50

(52) U.S. Cl. CPC A61B 8/403; A61B 6/461; A61B 6/502

(57) ABSTRACT

A control apparatus including at least one processor, wherein the processor is configured to: specify a first region of interest of a breast put into a compressed state between an imaging table and a compression member; perform control of rotating at least the compression member while the breast is put into the compressed state so that the first region of interest approaches the compression member; and perform control of capturing an ultrasound image of the first region of interest via the rotated compression member.

### CROSS-REFERENCE TO RELATED APPLICATIONS

This application claims priority from Japanese Application No. 2023-038161, filed on Mar. 10, 2023, the entire disclosure of which is incorporated herein by reference.

### BACKGROUND

### Technical Field

The present disclosure relates to a control apparatus, a control method, and a control program.

### Related Art

In the related art, a mammography apparatus that captures a radiation image of a breast is known. In addition, in the terms of improving the detection accuracy of a lesion and improving the efficiency of an examination, an apparatus that can capture an ultrasound image of a breast in addition to a radiation image is proposed. For example, JP2017-176509A discloses an apparatus that captures a radiation image and an ultrasound image of a breast put into a compressed state by a compression member. In the apparatus described in JP2017-176509A, an ultrasound probe is moved along an upper surface (surface opposite to a surface on which the breast of an examinee is disposed) of the compression member. In addition, for example, JP2020-000446A discloses a technique in which a transducer group that receives ultrasound is provided in a compression plate, and a pressure in the compression plate is adjusted to increase a contact area in contact with a breast of an examinee.

By the way, in the ultrasound image, according to the principle of irradiating an object with the ultrasound and receiving the reflected waves, the image is unclear in a case in which there is a void between an ultrasound emitting unit and the object. Therefore, in a case in which the breast in the compressed state is subjected to ultrasound imaging, it is desired that the compression member and the breast are closely attached to each other so as not to generate the void. However, since the breast is thickest in a central portion, even in a case in which the breast is compressed by the compression member, the void may be generated between the breast and the compression member, particularly at the end portion. In a case in which a portion suspected to be abnormal is located at an end portion of the breast, an appropriate image diagnosis may not be possible.

### SUMMARY

The present disclosure provides a control apparatus, a control method, and a control program that can obtain, even in a case in which a portion suspected to be abnormal is located at an end portion of a breast, an ultrasound image in which an influence of a void between a compression member and the breast is reduced as compared to a case in which the present disclosure is not implemented.

A first aspect of the present disclosure relates to a control apparatus comprising: at least one processor, in which the processor specifies a first region of interest of a breast put into a compressed state between an imaging table and a compression member, performs control of rotating at least the compression member while the breast is put into the compressed state so that the first region of interest approaches the compression member, and performs control of capturing an ultrasound image of the first region of interest via the rotated compression member.

In the first aspect, the processor may determine whether or not the first region of interest is in contact with the compression member, and may perform control of rotating the compression member so that the first region of interest approaches the compression member in a case in which it is determined that the first region of interest is not in contact with the compression member.

In the first aspect, the processor may acquire the ultrasound image of the first region of interest, and may determine whether or not the first region of interest is in contact with the compression member based on the ultrasound image.

In the first aspect, the processor may determine that the first region of interest is not in contact with the compression member in a case in which a portion in which a signal value is changed discontinuously exists in the ultrasound image of the first region of interest.

In the first aspect, the processor may acquire a radiation image of the breast put into the compressed state, may specify a position of a second region of interest in the radiation image, and may specify a region of the breast corresponding to the second region of interest as the first region of interest.

In the first aspect, the processor may perform control of displaying the radiation image on a display, and may receive designation of the position of the second region of interest in the radiation image.

In the first aspect, the processor may extract the second region of interest in the radiation image.

In the first aspect, the processor may perform control of rotating the compression member around an axis extending from a chest wall side of the breast to a nipple side.

A second aspect of the present disclosure relates to a control method comprising: specifying a first region of interest of a breast put into a compressed state between an imaging table and a compression member; performing control of rotating at least the compression member while the breast is put into the compressed state so that the first region of interest approaches the compression member; and performing control of capturing an ultrasound image of the breast including the first region of interest via the rotated compression member.

A third aspect of the present disclosure relates to a control program for causing a computer to execute a process comprising: specifying a first region of interest of a breast put into a compressed state between an imaging table and a compression member; performing control of rotating at least the compression member while the breast is put into the compressed state so that the first region of interest approaches the compression member; and performing control of capturing an ultrasound image of the breast including the first region of interest via the rotated compression member.

According to the aspects described above, in the control apparatus, the control method, and the control program according to the present disclosure, it is possible to obtain, even in a case in which the portion suspected to be abnormal is located at the end portion of the breast, the ultrasound image in which the influence of the void between the compression member and the breast is reduced as compared to a case in which the present disclosure is not implemented.

### BRIEF DESCRIPTION OF THE DRAWINGS

FIG. 1 is a view showing an example of a schematic configuration of an imaging system.

FIG. 2 is a side view showing an example of an appearance of an imaging apparatus.

FIG. 3 is a three-orthographic view showing an example of a schematic configuration of a compression member.

FIG. 4 is a three-orthographic view showing an example of the schematic configuration of the compression member.

FIG. 5 is a view showing rotation of the compression member.

FIG. 6 is a view showing the rotation of the compression member.

FIG. 7 is a block diagram showing an example of a hardware configuration of a console.

FIG. 8 is a block diagram showing an example of a functional configuration of the console.

FIG. 9 is a view showing an example of a first region of interest.

FIG. 10 is a view showing an example of a screen displayed on a display.

FIG. 11 is a view showing an example of a good quality ultrasound image.

FIG. 12 is a view showing an example of a bad quality ultrasound image.

FIG. 13 is a flowchart showing an example of a control process according to a first example.

FIG. 14 is a flowchart showing an example of a control process according to a second example.

FIG. 15 is a flowchart showing an example of a control process according to a third example.

FIG. 16 is a view showing rotation of the compression member and an imaging table.

### DETAILED DESCRIPTION

Hereinafter, a description of an embodiment of the present disclosure will be made with reference to the accompanying drawings.

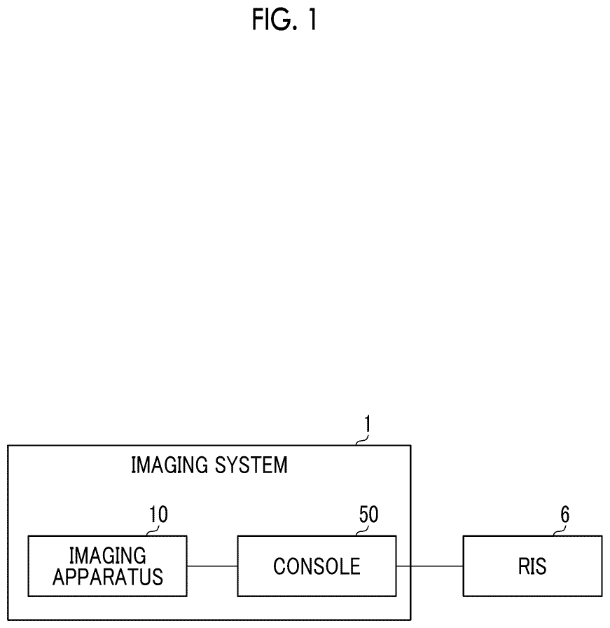

First, a description of a configuration of an imaging system 1 will be made with reference to FIG. 1. FIG. 1 is a view showing an example of a schematic configuration of the imaging system 1. As shown in FIG. 1, the imaging system 1 comprises an imaging apparatus 10 and a console 50. The imaging apparatus 10 and the console 50, and the console 50 and an external radiology information system (RIS) 6 are configured to be connected to each other via a wired or wireless network.

In the imaging system 1, the console 50 acquires an imaging order or the like from the RIS 6, and controls the imaging apparatus 10 in accordance with the imaging order, an instruction from the user, and the like. The imaging apparatus 10 acquires a radiation image and an ultrasound image of a breast of an examinee put into a compressed state by a compression member 40 as a subject. The console 50 is an example of a control apparatus according to the present disclosure.

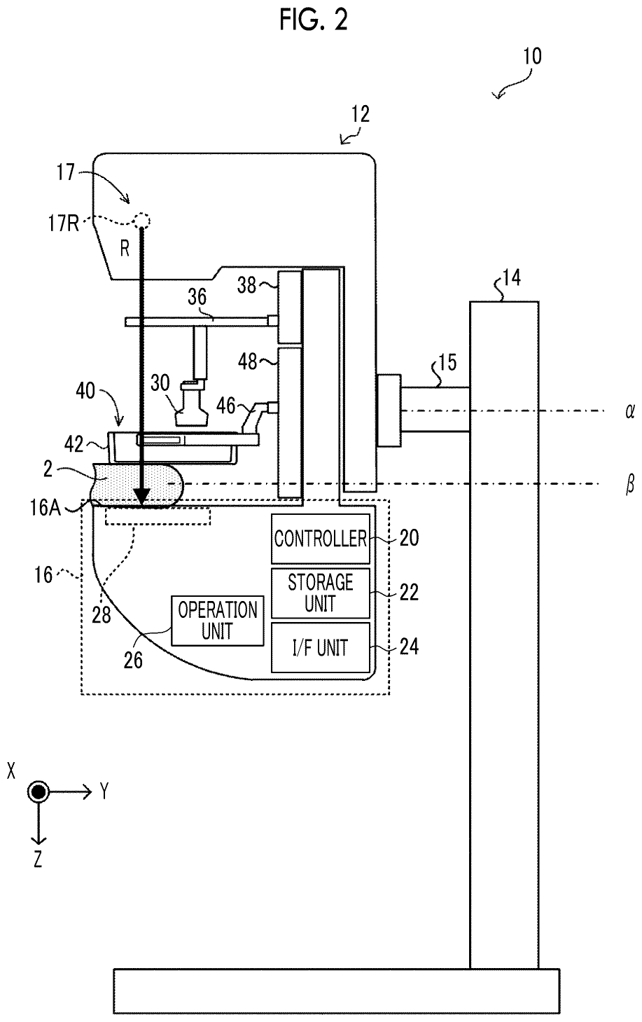

Next, a description of a schematic configuration of the imaging apparatus 10 will be made with reference to FIG. 2. FIG. 2 is a side view showing an example of an appearance of the imaging apparatus 10, and is a view in a case in which the imaging apparatus 10 is viewed from a right side of the examinee. As shown in FIG. 2, the imaging apparatus 10 comprises a radiation source 17R, a radiation detector 28, an imaging table 16 disposed between the radiation source 17R and the radiation detector 28, the compression member 40 that compresses a breast 2 between the compression member 40 and the imaging table 16, and an ultrasound probe 30 disposed between the radiation source 17R and the compression member 40. In the imaging apparatus 10, a user, such as a doctor or a technician, positions the breast 2 of the examinee on an imaging surface 16A of the imaging table 16.

The imaging apparatus 10 comprises an arm part 12, a base 14, and a shaft part 15. The arm part 12 is held to be movable in an up-down direction (Z direction) by the base 14. The shaft part 15 connects the arm part 12 to the base 14. The arm part 12 is relatively rotatable with respect to the base 14 with the shaft part 15 as a rotation axis a. In addition, the arm part 12 may be relatively rotatable with respect to the base 14 with the shaft part 15 as the rotation axis a separately between an upper part comprising a radiation emitting unit 17 and a lower part comprising the imaging table 16.

The arm part 12 comprises the radiation emitting unit 17 and the imaging table 16. The radiation emitting unit 17 comprises the radiation source 17R, and is configured to change an irradiation field of radiation (for example, X-rays) emitted from the radiation source 17R. For example, the change of the irradiation field may be performed by the user operating an operation unit 26, or may be performed by a controller 20 in accordance with a type of the attached compression member 40. The radiation source 17R irradiates the breast put into the compressed state by the compression member 40 with radiation R.

The imaging table 16 comprises the controller 20, a storage unit 22, an interface (I/F) unit 24, the operation unit 26, and the radiation detector 28. The controller 20 controls an overall operation of the imaging apparatus 10 in accordance with the control of the console 50. The controller 20 comprises a central processing unit (CPU), a read only memory (ROM), a random access memory (RAM), and the like (none shown). The ROM stores in advance various programs including a program executed by the CPU for performing the control related to the acquisition of the radiation image and the ultrasound image. The RAM transitorily stores various data.

Data of the radiation image and the ultrasound image, various types of other information, and the like are stored in the storage unit 22. The storage unit 22 is realized by, for example, a storage medium, such as a hard disk drive (HDD), a solid state drive (SSD), and a flash memory.

The I/F unit 24 performs communication of various types of information with the console 50 by wired or wireless communication. Specifically, the I/F unit 24 receives information related to the control of the imaging apparatus 10 from the console 50. Further, the I/F unit 24 transmits the data of the radiation image and the ultrasound image to the console 50.

The operation unit 26 is a part that is provided on the imaging table 16 or the like and can be operated by the user with a hand, a foot, or the like, and is, for example, a switch, a button, or a touch panel. For example, the operation unit 26 may receive a voice input from the user.

The radiation detector 28 is disposed in the imaging table 16, detects the radiation R transmitted through the breast and the imaging table 16, generates the radiation image based on the detected radiation R, and outputs image data indicating the generated radiation image. It should be noted that a type of the radiation detector 28 is not particularly limited and may be, for example, an indirect conversion type radiation detector that converts the radiation R into light and converts the converted light into a charge, or a direct conversion type radiation detector that directly converts the radiation R into a charge.

A probe unit 38 and a compression unit 48 are connected to the arm part 12. A support part 36 that attachably and detachably supports the ultrasound probe 30 is attached to the probe unit 38. The support part 36 (ultrasound probe 30) is moved in the up-down direction (Z direction) and a horizontal direction (X direction and Y direction) by a driving unit (not shown) provided in the probe unit 38. In addition, the support part 36 may be relatively rotatable with respect to the base 14 with an engaging part with the probe unit 38 as a rotation axis. It is preferable that the support part 36 is formed of a material that transmits the radiation R.

The ultrasound probe 30 is used to obtain the ultrasound image of the breast put into the compressed state by the compression member 40, is disposed between the radiation source 17R and the compression member 40, irradiates the breast with ultrasound via the compression member 40, and receives the reflected waves from the breast. Specifically, the ultrasound probe 30 comprises an ultrasound transducer array. The ultrasound transducer array is configured such that a plurality of ultrasound transducers are arranged one-dimensionally or two-dimensionally. The ultrasound transducer is formed, for example, such that electrodes are formed on both ends of a piezoelectric body, such as a piezoelectric ceramic represented by lead (Pb) zirconate titanate (PZT) or a polymer piezoelectric element represented by polyvinylidene difluoride (PVDF). The probe unit 38 includes a converter (not shown) that converts the reflected waves from the breast received by the ultrasound probe 30 into the ultrasound image, and the ultrasound image is obtained by the converter.

In addition, a plurality of types of the ultrasound probes 30 different from each other may be attachable to the imaging apparatus 10. For example, depending on a physique of the examinee (for example, a size of the breast), a tissue composition of the breast (for example, a fat mass and a mammary gland mass), a type of imaging (for example, magnification imaging and spot imaging), and the like, the ultrasound probes 30 having different types from each other may be prepared and can be attached to and detached from the imaging apparatus 10. For example, the ultrasound probes 30 having different performances and dimensions from each other may be selectively used, such as a linear probe having a center frequency of about 7.5 MHz (for superficial use or the like), a convex probe having a center frequency of about 3.5 MHz (for abdomen or the like), and a sector probe having a center frequency of about 2.5 MHz (for heart or the like).

A support part 46 that supports the compression member 40 is attachably and detachably attached to the compression unit 48. The support part 46 (compression member 40) is moved in the up-down direction (Z direction) by a driving unit (not shown) provided in the compression unit 48. In addition, the support part 46 may be relatively rotatable with respect to the base 14 with an engaging part with the compression unit 48 as a rotation axis. That is, the compression member 40 may be rotatable around an axis (axis extending in the Y direction) extending from a chest wall side of the breast to a nipple side.

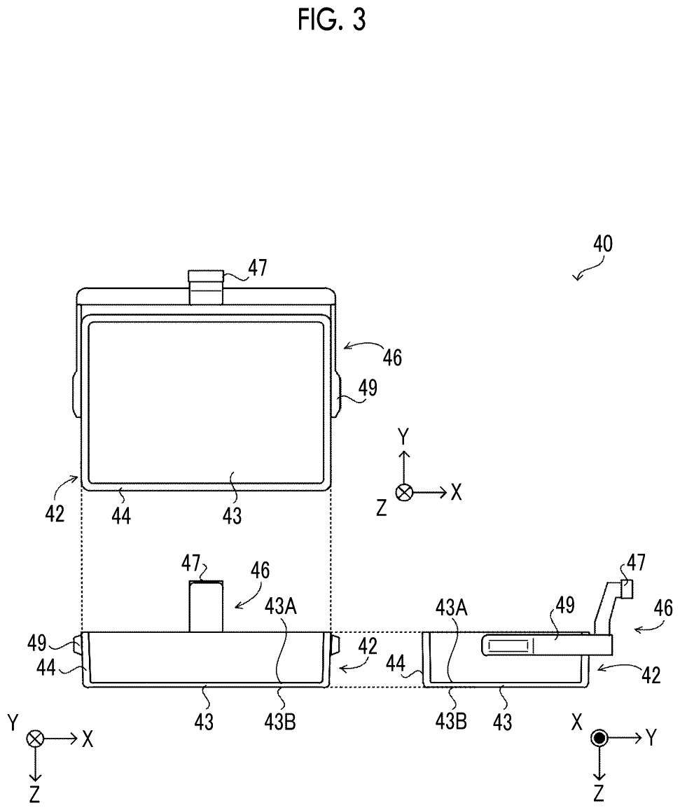

The compression member 40 is used to put the breast disposed on the imaging surface 16A into the compressed state. Specifically, the compression member 40 is disposed between the radiation source 17R and the imaging table 16 and interposes the breast between the compression member 40 and the imaging table 16 to put the breast into the compressed state. FIG. 3 shows a three-orthographic view of an example of the compression member 40. The three-orthographic view of FIG. 3 includes a top view of the compression member 40 as viewed from above (radiation emitting unit 17 side), a side view thereof as viewed from the examinee side, and a side view thereof as viewed from the right side of the examinee. As shown in FIG. 3, the compression member 40 includes a compression part 42 and the support part 46.

The support part 46 includes an attachment part 47 and an arm 49. The attachment part 47 attaches the compression member 40 to the imaging apparatus 10, specifically, the driving unit of the compression unit 48. The arm 49 supports the compression part 42.

The compression part 42 includes a bottom part 43 formed to be substantially flat and surrounded by a wall part 44 having a substantially uniform height, and has a cross section shape formed in a recess shape. It is preferable that the compression part 42 is formed of an optically transparent or translucent material in order to perform positioning and check of the compressed state in the compression of the breast. In addition, it is preferable that the compression part 42 is formed of a material excellent in a transmittance of the radiation R and the ultrasound. In addition, it is preferable that the compression part 42 is formed of, for example, a material excellent in strength, such as drop strength and compression strength.

As such a material, for example, resin, such as polymethylpentene (PMP), polycarbonate (PC), acryl, polypropylene (PP), and polyethylene terephthalate (PET), can be used. In particular, in the polymethylpentene, an acoustic impedance, which affects the transmittance and the reflectivity of the ultrasound, is closer to an acoustic impedance of a human body (breast) than other materials, and a proportion of the noise on the ultrasound image can be decreased. Therefore, as the material of the compression part 42, the polymethylpentene is suitable.

In addition, a plurality of types of the compression members 40 different from each other may be attachable to the imaging apparatus 10. For example, depending on a physique of the examinee (for example, a size of the breast), a tissue composition of the breast (for example, a fat mass and a mammary gland mass), a type of imaging (for example, magnification imaging and spot imaging), and the like, compression members 40 having different types from each other may be prepared and can be attached to and detached from the imaging apparatus 10. Specifically, a compression member in accordance with the size of the breast, a compression member for axilla imaging, a compression member for magnification imaging, and a compression member for so-called spot imaging that captures the radiation image of only a region in which a lesion exists, and the like may be used. That is, the compression member 40 is not limited to the compression member that compresses the entire breast, and may have a smaller size than the breast to compress a part of the breast.

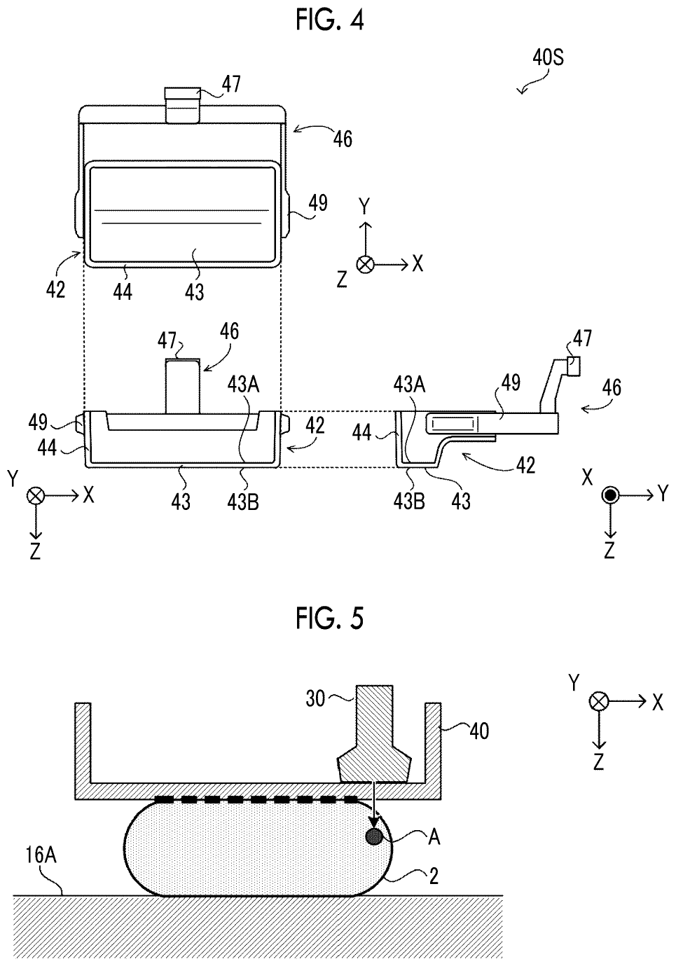

FIG. 4 shows a three-orthographic view of a compression member 40S for a small breast as an example of another form different from the compression member 40 of FIG. 3. The three-orthographic view of FIG. 4 includes a top view of the compression member 40S as viewed from above (radiation emitting unit 17 side), a side view thereof as viewed from the examinee side, and a side view thereof as viewed from the right side of the examinee. The compression member 40S includes the compression part 42 and the support part 46, as in the compression member 40 in FIG. 3. In the compression member 40S, the bottom part 43 is not flat, and the attachment part 47 side is higher than a chest wall side (side away from the attachment part 47). In addition, the height of the wall part 44 is not uniform, and a height of a part of the chest wall side is lower than a height of other parts. Due to such a shape, the compression member 40S can easily perform the positioning and the compression even for the small breast.

As described above, in the imaging apparatus 10, at least one of the compression member 40 for putting the breast into the compressed state or the ultrasound probe 30 for acquiring the ultrasound image may be attachable and detachable. That is, a plurality of types of the compression members 40 and the ultrasound probes 30 having different dimensions from each other may be attachable to the imaging apparatus 10. In this case, the imaging apparatus 10 may detect the types of the compression member 40 and the ultrasound probe 30 that are attached.

For example, the attachment part 47 of the compression member 40 may be provided with a plurality of pins having different dispositions for each type of the compression member 40 as identification information, and the identification information may be read by a sensor (for example, a photointerrupter) that can detect the disposition of the pins provided in the compression unit 48. In addition, for example, a marker (for example, a bar code and a two-dimensional code) in accordance with the type of the compression member 40 may be provided at any position of the compression member 40 as identification information, and the identification information may be read by a sensor (for example, a charge coupled device (CCD) sensor) that can detect the marker.

In addition, for example, a radio frequency identification (RFID) tag having identification information in accordance with the type of the compression member 40 may be provided at any position of the compression member 40, and the identification information may be read by an RFID reader that can read the RFID tag. In addition, for example, a weight of each type of the compression member 40 and identification information may be stored in the storage unit 22 in advance in association with each other, the weight of the attached compression member 40 may be measured by a sensor that can detect the weight, and the identification information (type of the compression member 40) may be specified based on a measured value.

Similarly, for the ultrasound probe 30, the type of the attached ultrasound probe 30 may be identified in accordance with, for example, the pin, the marker, the RFID tag, or the weight.

It should be noted that a gel-like or liquid medium having an ultrasound transmittance may be applied to an upper surface 43A of the bottom part 43 of the compression member 40 and/or a contact surface 43B with the breast. As such a medium, for example, a known jelly for an ultrasound examination, which has the acoustic impedance close to the acoustic impedance of the human body (breast), can be applied. That is, the imaging apparatus 10 may acquire the ultrasound image of the breast put into the compressed state by the compression member 40 in a state of being coated with the gel-like or liquid medium having the ultrasound transmittance, via the compression member 40. In this case, it is possible to suppress entry of air into an interface between an ultrasound radiation surface of the ultrasound probe 30 and the upper surface 43A and/or an interface between the contact surface 43B and the breast, and it is possible to reduce a difference in the acoustic impedance at each interface, so that the proportion of the noise applied to the ultrasound image can be decreased.

It should be noted that the method of imaging the breast via the imaging apparatus 10 is not particularly limited. For example, cranio-caudal (CC) imaging, medio-lateral oblique (MLO) imaging, the magnification imaging and the spot imaging for imaging a part of the breast, and the like may be performed. The CC imaging is a method of imaging the breast in the compressed state by interposing the breast between the imaging table 16 and the compression member 40 in the up-down direction (Z direction). The MLO imaging is a method of imaging the breast in the compressed state including an axilla portion by interposing the breast between the imaging table 16 and the compression member 40 in a tilted state in which a rotation angle of the arm part 12 with respect to the base 14 is equal to or greater than 45 degrees and smaller than 90 degrees.

In addition, for example, the imaging apparatus 10 may perform tomosynthesis imaging. In the tomosynthesis imaging, the radiation R is emitted from each of a plurality of irradiation positions having different irradiation angles toward the breast by the radiation source 17R, to capture a plurality of radiation images of the breast. That is, in the tomosynthesis imaging, the imaging is performed by changing the rotation angle of the radiation emitting unit 17 with respect to the base 14 while fixing the angles of the imaging table 16, the compression member 40, the breast, and the like.

In addition, in the imaging apparatus 10, the breast of the examinee may be positioned not only in a state in which the examinee is standing (standing state) but also in a state in which the examinee is sitting on a chair, a wheelchair, or the like (sitting state).

By the way, in the ultrasound image, according to the principle of irradiating an object with the ultrasound and receiving the reflected waves, the image is unclear in a case in which there is a void between the ultrasound probe 30 and the object. Therefore, in a case in which the breast 2 in the compressed state is subjected to the ultrasound imaging, it is desired that the compression member 40 and the breast 2 are closely attached to each other so as not to generate the void.

However, the breast 2 has a shape in which the thickness is greatest in a central portion and is smaller toward an end portion. FIG. 5 is a schematic view showing a case in which the imaging apparatus 10 is viewed from the chest wall side of the examinee toward the nipple side, and a thick broken line indicates a portion in which the breast 2 and the compression member 40 are in contact with each other. As shown in FIG. 5, even in a case in which the breast 2 is compressed by the compression member 40, the void may be generated between the breast 2 and the compression member 40 at the end portion. In this case, in a case in which a portion suspected to be abnormal (region of interest A) is located at the end portion of the breast 2, an appropriate image diagnosis may not be possible due to the influence of the void.

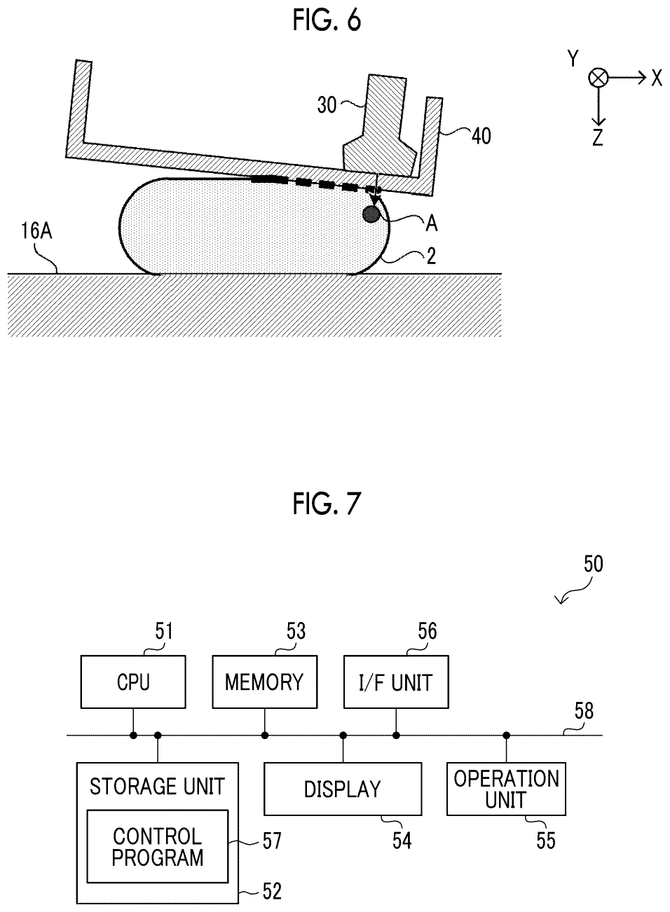

Then, as shown in FIG. 6, in a case in which the portion suspected to be abnormal (region of interest A) is located at the end portion of the breast 2, the console 50 according to the present disclosure performs control of rotating at least the compression member 40 so that a portion of the breast 2 including the region of interest A approaches the compression member 40. Accordingly, the ultrasound image is obtained in which the influence of the void between the compression member 40 and the breast 2 is reduced as compared to a case in which the present disclosure is not implemented. Hereinafter, a description of the console 50 will be made.

A description of an example of a hardware configuration of the console 50 will be made with reference to FIG. 7. As shown in FIG. 7, the console 50 includes a CPU 51, a non-volatile storage unit 52, and a memory 53 as a transitory storage region. In addition, the console 50 includes a display 54, such as a liquid crystal display, an operation unit 55, such as a touch panel, a keyboard, and a mouse, and an OF unit 56. The OF unit 56 performs wired or wireless communication with the imaging apparatus 10, the RIS 6, and other external apparatuses. The CPU 51, the storage unit 52, the memory 53, the display 54, the operation unit 55, and the OF unit 56 are connected to each other via a bus 58, such as a system bus and a control bus, so that various types of information can be exchanged.

The storage unit 52 is realized by, for example, a storage medium, such as an HDD, an SSD, and a flash memory. A control program 57 in the console 50 is stored in the storage unit 52. The CPU 51 reads out the control program 57 from the storage unit 52 to deploy the control program 57 into the memory 53, and executes the deployed control program 57. As the console 50, for example, a personal computer, a server computer, a smartphone, a tablet terminal, a wearable terminal, or the like can be applied as appropriate.

In addition, the storage unit 52 stores the image data of the radiation image and the ultrasound image acquired by the imaging apparatus 10, various types of other information, and the like. The image data of the radiation image and the ultrasound image may be stored in association with at least one of the imaging order or the imaging information. The imaging information may be, for example, at least one of examinee information and an imaging item that are included in the imaging order, photographer information indicating a photographer (for example, the user, such as the doctor or the technician) who performs the imaging, or date and time information indicating date and time when the imaging is performed.

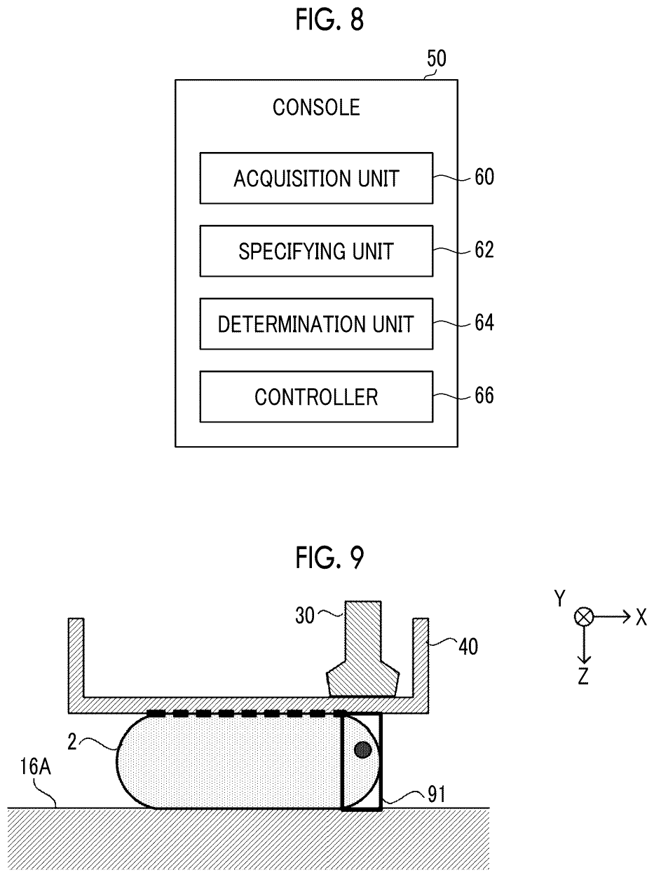

A description of an example of a functional configuration of the console 50 will be made with reference to FIG. 8. As shown in FIG. 8, the console 50 includes an acquisition unit 60, a specifying unit 62, a determination unit 64, and a controller 66. In a case in which the CPU 51 executes the control program 57, the CPU 51 functions as the acquisition unit 60, the specifying unit 62, the determination unit 64, and the controller 66.

### First Example

The acquisition unit 60 acquires a radiation image 90 of the breast 2 put into the compressed state from the imaging apparatus 10. Specifically, the acquisition unit 60 may acquire the radiation image stored in the storage unit 22 of the imaging apparatus 10 via the OF unit 56, may acquire the radiation image stored in the storage unit 52, or may acquire the radiation image stored in the external apparatus.

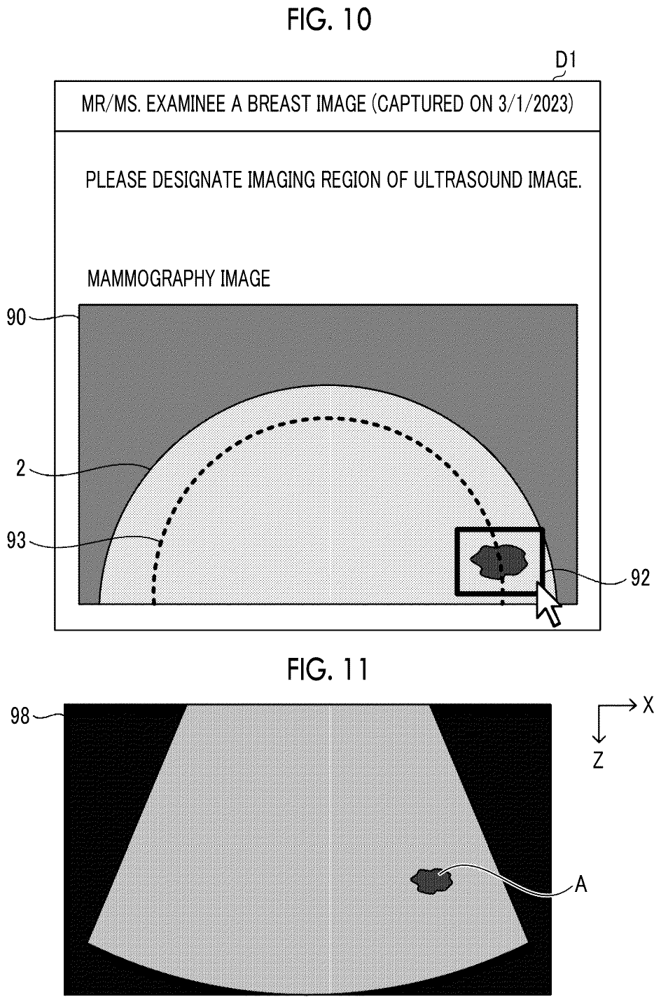

The specifying unit 62 specifies a first region of interest 91 of the breast 2 put into the compressed state between the imaging table 16 and the compression member 40. FIG. 9 shows an example of the first region of interest 91 specified for the breast 2. FIG. 9 is a schematic view showing a case in which the imaging apparatus 10 is viewed from the chest wall side of the examinee toward the nipple side, and a thick broken line indicates the portion in which the breast 2 and the compression member 40 are in contact with each other.

Specifically, the specifying unit 62 first specifies a position of a second region of interest 92 in the radiation image 90 acquired by the acquisition unit 60. The second region of interest 92 is, for example, a region of an abnormal shadow in which a tumor, a cancer, or the like is suspected, and a region having a high-density mammary gland (dense breast).

For example, the specifying unit 62 may specify a region designated by the user via the operation unit 55, as the second region of interest 92. FIG. 10 is an example of a screen D1 displayed on the display 54 by the controller 66, and includes the radiation image 90 (mammography image). As shown in FIG. 10, the controller 66 may perform control of displaying the radiation image 90 acquired by the acquisition unit 60 on the display 54, and may receive the designation of the position of the second region of interest 92 in the radiation image 90. In addition, in the radiation image 90 of FIG. 10, a contact region 93 in which the breast 2 and the compression member 40 are in contact with each other is indicated by a broken line.

In addition, for example, the specifying unit 62 may appropriately apply a method using a known computer aided detection/diagnosis (CAD) technique as the method of specifying the position of the second region of interest 92. As the method of specifying the region of interest using the CAD technique, for example, a method using a learning model, such as a convolutional neural network (CNN), can be applied. For example, the specifying unit 62 may extract the second region of interest 92 in the radiation image 90 by using a learning model trained to receive the radiation image as an input and then extract and output the region of interest included in the radiation image.

Next, the specifying unit 62 specifies a region of the breast 2 corresponding to the second region of interest 92 specified in the radiation image 90, as the first region of interest 91. The region of the breast 2 corresponding to the second region of interest 92 can be specified, for example, by associating coordinates in the radiation image 90 with the position of the bottom part 43 of the compression member 40.

The determination unit 64 determines whether or not the first region of interest 91 specified by the specifying unit 62 is in contact with the compression member 40. Specifically, the determination unit 64 acquires the ultrasound image by tentatively imaging the first region of interest 91 by using the ultrasound probe 30. The term “tentatively” is used because there may be the void between the first region of interest 91 of the breast 2 and the compression member 40 in this stage, and the good quality ultrasound image is not always obtained. Hereinafter, this tentative ultrasound imaging will be referred to as ultrasound pre-scanning.

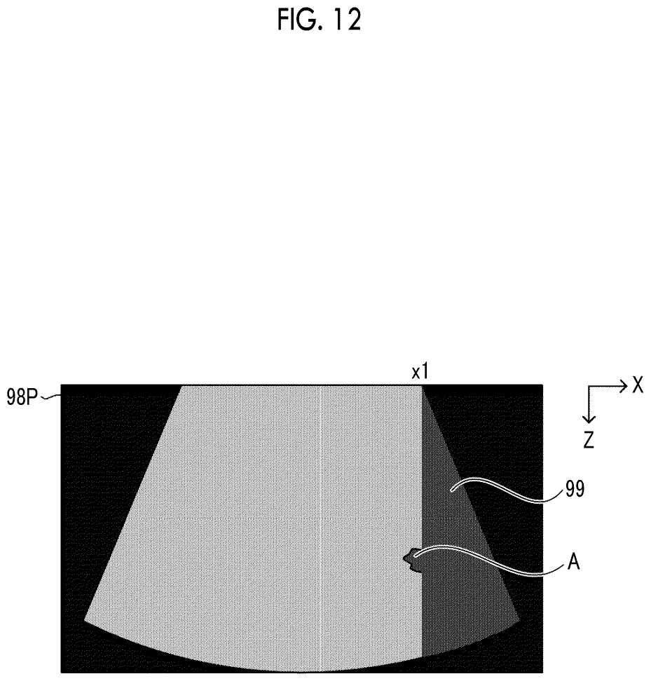

FIG. 11 shows an example of a good quality ultrasound image 98 in which the region of interest A is clear. FIG. 12 shows an example of a bad quality ultrasound image 98P in which the region of interest A is unclear. As shown in FIG. 12, in the bad quality ultrasound image 98P, the pixel value is steeply changed with a coordinate x1 at a certain position in the X direction as a boundary. This is because the pixel value is changed due to the influence of the void since the imaging is performed in a state in which there is the void between the first region of interest 91 and the compression member 40 (see FIG. 5). In a case in which there is the void between the breast 2 and the compression member 40, a portion 99 in which the signal value is changed discontinuously may be generated. In other words, by eliminating the void between the compression member 40 and the breast 2, the good quality ultrasound image 98 as shown in FIG. 11 can be obtained.

The determination unit 64 determines whether or not the first region of interest 91 is in contact with the compression member 40 based on the ultrasound image obtained by the ultrasound pre-scanning. For example, as described above, in a case in which there is the void between the breast 2 and the compression member 40, the portion 99 in which the signal value is changed discontinuously may be generated in the ultrasound image. Then, in a case in which the portion 99 in which the signal value is changed discontinuously exists in the ultrasound image of the first region of interest 91 obtained by the ultrasound pre-scanning, the determination unit 64 determines that the first region of interest 91 is not in contact with the compression member 40.

The controller 66 performs control of rotating at least the compression member 40 while the breast 2 is put into the compressed state so that the first region of interest 91 specified by the specifying unit 62 approaches the compression member 40. Specifically, in a case in which the determination unit 64 determines that the first region of interest 91 is not in contact with the compression member 40, the controller 66 performs control of rotating the compression member 40 so that the first region of interest 91 approaches the compression member 40. For example, as shown in FIG. 6, the first region of interest 91 approaches the compression member 40 by the controller 66 performing control of rotating the compression member 40 around the axis (axis extending in the Y direction) extending from the chest wall side of the breast to the nipple side.

The controller 66 controls the ultrasound probe 30 to capture the ultrasound image of the first region of interest 91 via the rotated compression member 40. In this case, since the ultrasound imaging can be performed in a state in which the first region of interest 91 is in contact with or is located near the compression member 40, the ultrasound image is obtained in which the influence of the void between the compression member 40 and the breast 2 is reduced.

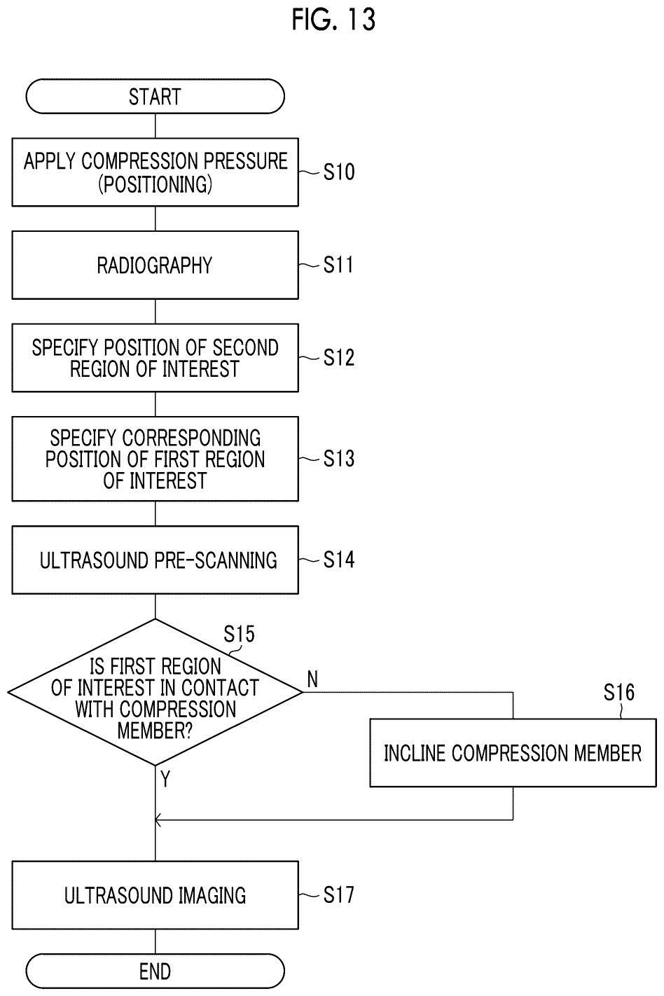

Next, a description of an action of the console 50 according to the present embodiment will be made with reference to FIG. 13. In the console 50, the CPU 51 executes the control program 57 to execute a control process shown in FIG. 13. The control process is executed, for example, in a case in which the user gives an instruction to start the execution via the operation unit 55.

In step S10, the controller 66 controls the imaging apparatus 10 to compress the breast 2 positioned by the doctor, the technician, and the like. In step S11, the controller 66 controls the imaging apparatus 10 to perform the radiography of the breast 2 put into the compressed state in step S10. In addition, the acquisition unit 60 acquires the radiation image 90 of the breast 2.

In step S12, the specifying unit 62 specifies the position of the second region of interest 92 in the radiation image 90 acquired in step S11. In step S13, the specifying unit 62 specifies the region of the breast 2 corresponding to the second region of interest 92 in the radiation image 90 specified in step S12, as the position of the first region of interest 91.

In step S14, the determination unit 64 acquires the ultrasound image by performing the ultrasound pre-scanning on the first region of interest 91 specified in step S13 by using the ultrasound probe 30. In step S15, the determination unit 64 determines whether or not the first region of interest 91 is in contact with the compression member 40 based on the ultrasound image obtained by the ultrasound pre-scanning and acquired in step S14.

In a case in which a negative determination is made in step S15, it is determined that the first region of interest 91 is not in contact with the compression member 40, and the process proceeds to step S16. In step S16, the controller 66 performs control of rotating at least the compression member 40 so that the first region of interest 91 specified in step S13 approaches the compression member 40, and the process proceeds to step S17. On the other hand, in a case in which an affirmative determination is made in step S15, it is determined that the first region of interest 91 is in contact with the compression member 40, and the process proceeds to step S17 without rotating the compression member 40.

In step S17, the controller 66 controls the ultrasound probe 30 to capture the ultrasound image of the first region of interest 91 via the compression member 40, and terminates the present control process.

### Second Example

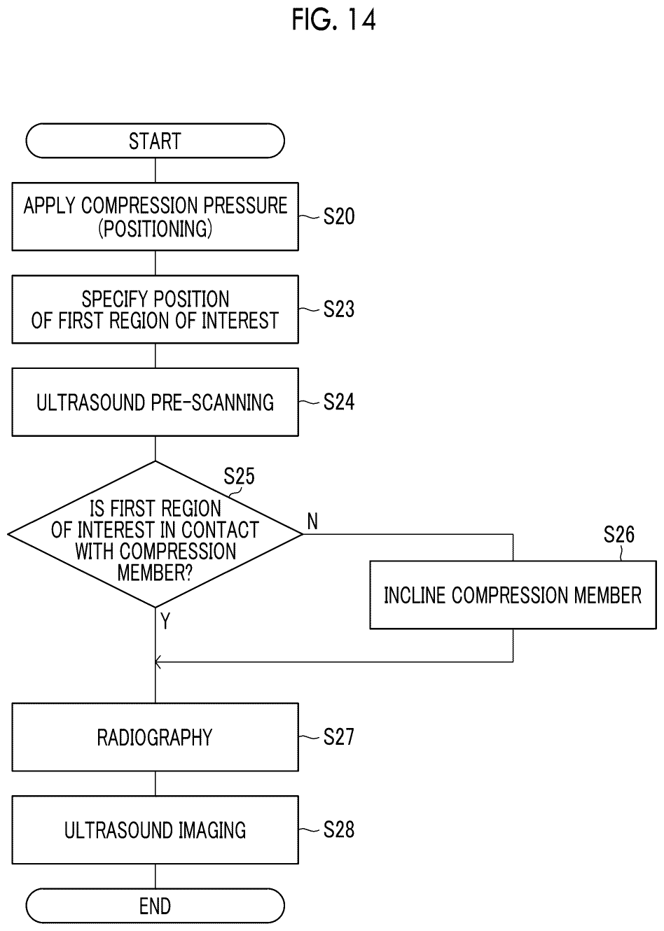

Although an example of the function and the action of the console 50 is described above, the console 50 according to the present disclosure can be variously modified. Hereinafter, a description of the second example will be made with reference to FIG. 14, but the description overlapping the first example will be partially omitted. In the first example, the form is described in which the ultrasound pre-scanning and the ultrasound imaging are performed after the radiography is performed. However, in the second example, the radiography and the ultrasound imaging are performed after it is checked whether or not the breast 2 and the compression member 40 are in contact with each other by the ultrasound pre-scanning.

In the console 50, the CPU 51 executes the control program 57 to execute a control process according to the second example shown in FIG. 14. The control process is executed, for example, in a case in which the user gives an instruction to start the execution via the operation unit 55.

In step S20, the controller 66 controls the imaging apparatus 10 to compress the breast 2 positioned by the doctor, the technician, and the like. In step S23, the specifying unit 62 specifies the position of the first region of interest 91 in the breast 2. In this case, for example, in a case in which an examination target portion (for example, an axilla) is determined in the imaging order and the like, the part can be applied as the first region of interest 91.

In step S24, the determination unit 64 acquires the ultrasound image by performing the ultrasound pre-scanning on the first region of interest 91 specified in step S23 by using the ultrasound probe 30. In step S25, the determination unit 64 determines whether or not the first region of interest 91 is in contact with the compression member 40 based on the ultrasound image obtained by the ultrasound pre-scanning and acquired in step S24.

In a case in which a negative determination is made in step S25, it is determined that the first region of interest 91 is not in contact with the compression member 40, and the process proceeds to step S26. In step S26, the controller 66 performs control of rotating at least the compression member 40 so that the first region of interest 91 specified in step S23 approaches the compression member 40, and the process proceeds to step S27. On the other hand, in a case in which an affirmative determination is made in step S25, it is determined that the first region of interest 91 is in contact with the compression member 40, and the process proceeds to step S27 without rotating the compression member 40.

In step S27, the controller 66 controls the imaging apparatus 10 to perform the radiography of the breast 2 put into the compressed state in step S20. In step S28, the controller 66 controls the ultrasound probe 30 to capture the ultrasound image of the first region of interest 91 via the compression member 40, and terminates the present control process.

### Third Example

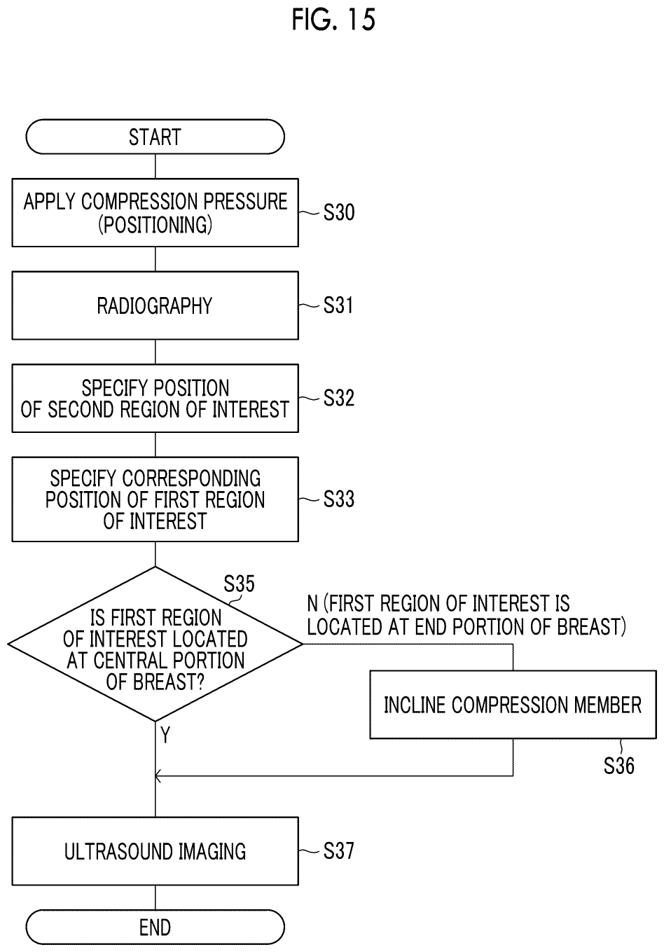

Next, a description of a third example will be made with reference to FIG. 15. As described above, in a case in which an attempt is made to perform the ultrasound imaging on the end portion of the breast 2, there is a high possibility that the obtained ultrasound image is unclear since the breast 2 and the compression member 40 are not closely attached to each other. Therefore, in the third example, in a case in which the first region of interest 91 is located at the end portion of the breast 2, control of rotating the compression member 40 is performed even without performing the determination of the contact with the compression member 40.

In the console 50, the CPU 51 executes the control program 57 to execute a control process according to the third example shown in FIG. 15. The control process is executed, for example, in a case in which the user gives an instruction to start the execution via the operation unit 55.

In step S30, the controller 66 controls the imaging apparatus 10 to compress the breast 2 positioned by the doctor, the technician, and the like. In step S31, the controller 66 controls the imaging apparatus 10 to perform the radiography of the breast 2 put into the compressed state in step S30. In addition, the acquisition unit 60 acquires the radiation image 90 of the breast 2.

In step S32, the specifying unit 62 specifies the position of the second region of interest 92 in the radiation image 90 acquired in step S31. In step S33, the specifying unit 62 specifies the region of the breast 2 corresponding to the second region of interest 92 in the radiation image 90 specified in step S32, as the position of the first region of interest 91. In step S35, the determination unit 64 determines whether the position of the first region of interest 91 specified in step S33 is located at the central portion or the end portion of the breast 2.

In a case in which a negative determination is made in step S35, it is determined that the first region of interest 91 is located at the end portion of the breast 2 and is unlikely to be in contact with the compression member 40, and the process proceeds to step S36. In step S36, the controller 66 performs control of rotating at least the compression member 40 so that the first region of interest 91 specified in step S33 approaches the compression member 40, and the process proceeds to step S37. On the other hand, in a case in which an affirmative determination is made in step S35, it is determined that the first region of interest 91 is located at the central portion of the breast 2 and the first region of interest 91 is likely to be in contact with the compression member 40, and the process proceeds to step S37 without rotating the compression member 40.

In step S37, the controller 66 controls the ultrasound probe 30 to capture the ultrasound image of the first region of interest 91 via the compression member 40, and terminates the present control process.

As described above, the console 50 according to the aspect of the present disclosure comprises at least one processor, in which the processor specifies the first region of interest 91 including at least a part of the breast 2 put into the compressed state between the imaging table 16 and the compression member 40, performs control of rotating at least the compression member 40 so that the first region of interest 91 approaches the compression member 40, and performs control of capturing the ultrasound image of the first region of interest 91 via the rotated compression member 40.

That is, with the console 50 of the present disclosure, even in a case in which the portion (first region of interest 91) suspected to be abnormal is located at the end portion of the breast 2, the ultrasound imaging can be performed in a state in which the first region of interest 91 is in contact with or is located near the compression member 40. Therefore, it is possible to obtain the ultrasound image in which the influence of the void between the compression member 40 and the breast 2 is reduced as compared to a case in which the technique of the present disclosure is not implemented.

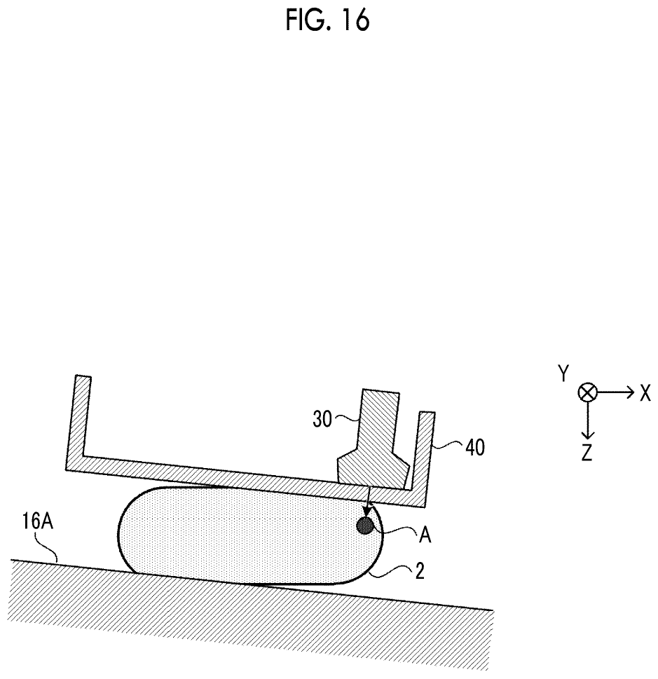

It should be noted that, in the embodiment described above, as shown in FIG. 6 and the like, the form is described in which only the compression member 40 is rotated to approach the first region of interest 91 and the imaging table 16 is not rotated, but the present disclosure is not limited to this. For example, as shown in FIG. 16, the compression member 40 and the imaging table 16 may be integrally rotated. For example, the entire arm part 12 may be rotated.

In a case in which the imaging table 16 and/or the arm part 12 is rotated, usually, the rotation is performed with the shaft part 15 as the rotation axis a as shown in FIG. 2. In this case, a load on the breast 2 is increased. Then, in a case in which the imaging table 16 and/or the arm part 12 is rotated, it is preferable that the rotation around the rotation axis a and the movement in the up-down direction (Z direction) by the base 14 are simultaneously performed so that the rotation is performed with a center β of the breast 2 as the rotation axis. It should be noted that the center β of the breast 2 can be derived by, for example, a sensor that can measure the compression thickness of the breast 2.

In addition, in the embodiment described above, the form is described in which the compression member 40 is rotated around the axis (axis extending in the Y direction of FIG. 2) extending from the chest wall side of the breast 2 to the nipple side, but the direction of the rotation axis is not particularly limited. Specifically, the compression member 40 may be configured to rotate around an axis (axis extending in the X direction in FIG. 2) extending to the left and right of the breast 2. For example, by rotating the compression member 40 around the axis extending to the left and right of the breast 2, the compression member 40 can be caused to approach the vicinity of the nipple (that is, the chest wall side can be lifted). Therefore, even in a case in which the first region of interest 91 is located in the vicinity of the nipple, it is possible to obtain the ultrasound image in which the influence of the void between the compression member 40 and the breast 2 is reduced.

In addition, specifically, the compression member 40 may be configured to rotate around an axis (axis extending in the Z direction of FIG. 2) extending in the compression direction of the breast 2. For example, in the MLO imaging, the compression member 40 can be fitted in a form that is swallowed into the axilla by rotating the compression member 40 around the axis extending in the compression direction of the breast 2. Therefore, even in a case in which the first region of interest 91 is located in the vicinity of the axilla, it is possible to obtain the ultrasound image in which the influence of the void between the compression member 40 and the breast 2 is reduced. In addition, the rotations around the respective axes may be combined.

In addition, in the embodiment described above, the form is described in which the console 50 is an example of a control apparatus according to the present disclosure, but an apparatus other than the console 50 may have the function of the control apparatus according to the present disclosure. In other words, an apparatus other than the console 50, such as the imaging apparatus 10 and the external apparatus, may have a part or all of the functions of the acquisition unit 60, the specifying unit 62, the determination unit 64, and the controller 66.

In the embodiment described above, for example, as hardware structures of processing units that execute various types of processes, such as the controller 20, the acquisition unit 60, the specifying unit 62, the determination unit 64, and the controller 66, various processors shown below can be used. As described above, in addition to the CPU that is a general-purpose processor that executes software (program) to function as various processing units, the various processors include a programmable logic device (PLD) that is a processor of which a circuit configuration can be changed after manufacture, such as a field programmable gate array (FPGA), and a dedicated electric circuit that is a processor having a circuit configuration that is designed for exclusive use in order to execute a specific process, such as an application specific integrated circuit (ASIC).

One processing unit may be configured by using one of the various processors or may be configured by using a combination of two or more processors of the same type or different types (for example, a combination of a plurality of FPGAs or a combination of a CPU and an FPGA). Moreover, a plurality of processing units may be configured of one processor.

A first example of the configuration in which the plurality of processing units are configured by using one processor is a form in which one processor is configured by using a combination of one or more CPUs and the software and this processor functions as the plurality of processing units, as represented by computers, such as a client and a server. Second, as represented by a system on chip (SoC) or the like, there is a form in which the processor is used in which the functions of the entire system which includes the plurality of processing units are realized by a single integrated circuit (IC) chip. In this way, as the hardware structure, the various processing units are configured by using one or more of the various processors described above.

Further, the hardware structure of these various processors is, more specifically, an electric circuit (circuitry) in which circuit elements such as semiconductor elements are combined.

In addition, in each embodiment described above, the aspect is described in which the various programs in the imaging apparatus 10 are stored (installed) in the ROM included in the controller 20 in advance, and the control program 57 in the console 50 is stored in the storage unit 52 in advance, but the present disclosure is not limited to this. The various programs and the control program 57 in the imaging apparatus 10 may be provided in a form of being recorded in a recording medium, such as a compact disc read only memory (CD-ROM), a digital versatile disc read only memory (DVD-ROM), and a universal serial bus (USB) memory. In addition, a form may be adopted in which the various programs and the control program 57 in the imaging apparatus 10 are downloaded from an external apparatus via the network. Further, the technique of the present disclosure extends to a storage medium that non-transitorily stores a program in addition to the program.

In the technique of the present disclosure, the embodiment and the examples described above can be combined as appropriate. The above-described contents and the above-shown contents are detailed description for parts according to the technique of the present disclosure, and are merely examples of the technique of the present disclosure. For example, the above description related to the configuration, the function, the action, and the effect is the description related to the examples of the configuration, the function, the action, and the effect of the parts according to the technique of the present disclosure. As a result, it is needless to say that unnecessary parts may be deleted, new elements may be added, or replacements may be made with respect to the above-described contents and the above-shown contents within a range that does not deviate from the gist of the technique of the present disclosure.

What is claimed is:

1. A control apparatus comprising at least one processor, wherein the processor is configured to:
specify a first region of interest of a breast put into a compressed state between an imaging table and a compression member;
perform control of rotating at least the compression member while the breast is put into the compressed state so that the first region of interest approaches the compression member; and
perform control of capturing an ultrasound image of the first region of interest via the rotated compression member.

2. The control apparatus according to claim 1, wherein the processor is configured to:
determine whether or not the first region of interest is in contact with the compression member; and
perform control of rotating the compression member so that the first region of interest approaches the compression member in a case in which it is determined that the first region of interest is not in contact with the compression member.

3. The control apparatus according to claim 2, wherein the processor is configured to:
acquire the ultrasound image of the first region of interest; and
determine whether or not the first region of interest is in contact with the compression member based on the ultrasound image.

4. The control apparatus according to claim 3, wherein the processor is configured to determine that the first region of interest is not in contact with the compression member in a case in which a portion in which a signal value is changed discontinuously exists in the ultrasound image of the first region of interest.

5. The control apparatus according to claim 1, wherein the processor is configured to:
acquire a radiation image of the breast put into the compressed state;
specify a position of a second region of interest in the radiation image; and
specify a region of the breast corresponding to the second region of interest as the first region of interest.

6. The control apparatus according to claim 5, wherein the processor is configured to:
perform control of displaying the radiation image on a display; and
receive designation of the position of the second region of interest in the radiation image.

7. The control apparatus according to claim 5, wherein the processor is configured to extract the second region of interest in the radiation image.

8. The control apparatus according to claim 1, wherein the processor is configured to perform control of rotating the compression member around an axis extending from a chest wall side of the breast to a nipple side.

9. A control method comprising:
specifying a first region of interest of a breast put into a compressed state between an imaging table and a compression member;
performing control of rotating at least the compression member while the breast is put into the compressed state so that the first region of interest approaches the compression member; and
performing control of capturing an ultrasound image of the first region of interest via the rotated compression member.

10. A non-transitory computer-readable storage medium storing a control program for causing a computer to execute a process comprising:
specifying a first region of interest of a breast put into a compressed state between an imaging table and a compression member;
performing control of rotating at least the compression member while the breast is put into the compressed state so that the first region of interest approaches the compression member; and
performing control of capturing an ultrasound image of the first region of interest via the rotated compression member.
